(19) United States
(12) Patent Application Publication
(10) Pub. No.: US 2025/0334203 A1
(43) Pub. Date: Oct. 30, 2025

(54) RESISTANCE VALVE

(71) Applicant: SOOCHOW UNIVERSITY, Wujiang (CN)

(72) Inventors: Peng Wu, Wujiang (CN); Liudi Zhang, Wujiang (CN); Yujiao Bai, Wujiang (CN); Xiangyu Zhao, Wujiang (CN); Xinyuan Mao, Wujiang (CN)

(21) Appl. No.: 19/260,835

(22) Filed: Jul. 7, 2025

Related U.S. Application Data
(63) Continuation of application No. PCT/CN2023/074209, filed on Feb. 2, 2023.

(30) Foreign Application Priority Data
Jan. 9, 2023 (CN) 202310027918.4

(51) Int. Cl. F16K 47/08; A61M 39/28; F16K 3/24

(52) U.S. Cl. CPC F16K 47/08; F16K 3/24; A61M 39/28

(57) ABSTRACT

The invention provides a resistance valve, including: an inner cylinder, where the inner cylinder can deform outward along a radial direction, a semi-elliptical outer spiral groove is formed on an outer wall of the inner cylinder, and an inclined deformation chute is provided inside the inner cylinder; a sliding block, an inclined surface of the sliding block abuts against the deformation chute, and the sliding block pushes the inner cylinder to deform outward; a rotating shaft, where the rotating shaft is in threaded connection to the sliding block, and the rotating shaft rotates to drive the sliding block to slide along an axial extension direction of the rotating shaft; and an outer cylinder, where a semi-elliptical inner spiral groove is opened on an inner wall of the outer cylinder body, and the inner spiral groove is matched with the outer spiral groove to form a plastic pipe accommodating space.

This application is a Continuation application of PCT/CN2023/074209, filed on Feb. 2, 2023, which claims priority to Chinse Patent Application No. 202310027918.4, filed on Jan. 9, 2023, which is incorporated by reference for all purposes as if fully set forth herein.

### FIELD OF THE INVENTION

The present disclosure relates to the fields of pharmaceuticals and biotechnology, and in particular to a resistance valve.

### DESCRIPTION OF THE RELATED ART

A resistance valve (back-pressure valve) can maintain the desired pressure in the pipeline, enabling normal output of flow from a pump. Back pressure, which occurs in all pump operations, refers to the pressure generated by the fluid output from the pump acting against the output direction. Typically, the resistance valve (back-pressure valve) is used during industrial liquid deliveries.

With the gradual clinical application of ventricular assist devices (also called blood pumps), higher safety requirements for blood pumps have been put forward. In vitro hemolysis test is a key link in evaluating the hemolytic performance of blood pumps. In the in vitro test platform for blood pumps, resistance valves are often used to compress medical tubing to locally form sudden constriction, which induce pressure loss to counteract the pressure head of blood pumps, thereby forming a loop that meets the testing conditions. However, the local flow field at the constriction is complex and includes recirculation zones, leading to a sharp increase in blood flow stress and significantly exacerbating blood damage. In addition to blood damage caused by the tested blood pump, blood damage induced by loop components, particularly resistance valves for calibrating back pressure, may affect the accuracy, repeatability, and comparability of hemolysis test results.

When extended to tubing for other fluids involving resistance valves (back-pressure valves), the impact of resistance valves on various indicators of the fluid cannot be ignored. Such valves can be used in loops for delivering sensitive fluids such as blood, macromolecular protein drugs, ultra-clean raw materials, and fuels. Especially in the biomedical industry, it is required to minimize the damage to the liquid during delivery, such as biopharmaceutical liquids, blood, and cell suspensions. However, components in the conveying loop have a certain impact on the fluid, so it is necessary to optimize some parts of the conveying pipe.

In the prior art, the patent publication No. WO2007107692A1 entitled “Valve” discloses a resistance valve for compressing a plastic pipe. According to the product, the lifting of the compression member is achieved by rotating a knob via threaded engagement, which variably flattens a plastic pipe placed on a concave surface of a base. The flattened pipe forms a localized constriction structure, where the flow of liquid is suddenly obstructed, thereby causing a back pressure to be provided in the pipeline. This mechanism maintains sufficient pressure in the entire loop, ensuring that the water pump operates normally and the liquid maintains steady flow through the pipe.

The resistance valve prepared by using the patent described above has the following disadvantages:
1. The conventional resistance valve described above forms a constriction structure with an excessively small cross-sectional area, which causes relatively large damage to blood. This substantially interferes with test results, making them unable to reflect the blood damage caused by the blood pump itself. As a result, the purpose of testing the performance of the blood pump itself cannot be achieved, leading to reduced detection efficiency, wasted experimental time, and consumption of experimental materials. When extended to broader fields, in addition to blood, sensitive fluids such as macromolecular protein drugs, ultra-clean raw materials, and fuels are all affected by the conventional resistance valve.
2. The resistance valve described above has a small adjustable pressure range, low adjustment precision, and slow response speed.
3. The resistance valve described above cannot store a large amount of liquid while adjusting pressure, thus failing to serve as a liquid reservoir.

### SUMMARY OF THE INVENTION

Therefore, the technical problem to be solved by the present disclosure is to overcome the defect in the prior art that a conventional resistance valve forms a constriction structure with an excessively small cross-sectional area, leading to significant blood damage. Provided is a resistance valve, which increases a clamping length of a plastic pipe by means of spiral winding and provides appropriate resistance to the plastic pipe by radial deformation of a cylinder, so as to provide back pressure required for the plastic pipe, thereby reducing the impact on a flowing medium in the plastic pipe.

In order to solve the technical problem described above, the present disclosure provides a resistance valve configured to provide resistance to a liquid in a plastic pipe, which includes:
an inner cylinder, where the inner cylinder is a cylindrical structure, the inner cylinder can deform outward along a radial direction, a semicircular outer spiral groove is formed on an outer wall of the inner cylinder, and an inclined deformation chute is provided inside the inner cylinder;
a sliding block provided in the inner cylinder, where the sliding block has an inclined surface matching the deformation chute, the sliding block slides in the inner cylinder along an extension direction of the deformation chute, the inclined surface of the sliding block abuts against the deformation chute, and the sliding block pushes the inner cylinder to deform outward along the radial direction;
a rotating shaft provided in the inner cylinder, where the rotating shaft extends through the sliding block and is in threaded connection to the sliding block, and the rotating shaft rotates to drive the sliding block to slide along an axial extension direction of the rotating shaft; and
an outer cylinder sleeved outside the inner cylinder, where a semicircular inner spiral groove is formed on an inner wall of the outer cylinder, and the inner spiral groove is matched with the outer spiral groove to form a multi-layer spiral plastic pipe accommodating space.

In one embodiment of the present disclosure, a plurality of deformation grooves are formed on a side wall of the inner cylinder, the deformation grooves are arranged along an axial extension direction of the inner cylinder, and the plurality of deformation grooves are evenly distributed around a circumference of the inner cylinder.

In one embodiment of the present disclosure, the outer cylinder is a cylindrical structure formed by splicing a plurality of arc-shaped sheets end to end, with a snap-fit structure provided between two adjacent arc-shaped sheets.

In one embodiment of the present disclosure, the rotating shaft partially protrudes beyond the inner cylinder, and a handle is provided at one end of the rotating shaft protruding beyond the inner cylinder.

In one embodiment of the present disclosure, the resistance valve further includes:
a bottom plate, where the inner cylinder and the outer cylinder are both provided on the bottom plate, the inner cylinder and the bottom plate are integrally formed, and the outer cylinder is snap-fitted onto the bottom plate;
an outer cylinder fixing ring sleeved outside the outer cylinder, where a retaining ring for limiting the outer cylinder fixing ring is formed on the bottom plate, and the outer cylinder fixing ring enables the outer cylinder to be in locked connection to the bottom plate;
a cover plate provided corresponding to the bottom plate and snap-fitted onto the inner cylinder and the outer cylinder, where a rotating shaft hole is formed on the cover plate; and
a connection limiting rod provided on the bottom plate and extending toward a direction of the cover plate, where the cover plate is in locked connection to the connection limiting rod by a bolt.

In one embodiment of the present disclosure, the outer cylinder fixing ring is provided with a fixing block, and an outer cylinder fixing ring slot is formed on the bottom plate; the outer cylinder fixing ring slot is provided corresponding to the fixing block in position, and the fixing block can be inserted into the outer cylinder fixing ring slot by rotating the outer cylinder fixing ring;
the outer cylinder fixing ring is further provided with a rotating handle.

In one embodiment of the present disclosure, the cover plate is further provided with an inner cylinder positioning block, and an inner cylinder positioning groove matching the inner cylinder positioning block is provided at a connection position between the inner cylinder and the cover plate.

In one embodiment of the present disclosure, the bottom plate and the cover plate are provided with an outer cylinder lower positioning block and an outer cylinder upper positioning block, respectively, and outer cylinder positioning grooves matching the outer cylinder lower positioning block and the outer cylinder upper positioning block are provided at connection positions between the outer cylinder and the bottom plate, and between the outer cylinder and the cover plate.

In one embodiment of the present disclosure, the bottom plate and the cover plate are respectively provided with a plurality of outer cylinder lower positioning blocks and outer cylinder upper positioning blocks, and the plurality of outer cylinder lower positioning blocks and outer cylinder upper positioning blocks are unevenly distributed.

Compared with the prior art, the above technical solutions of the present disclosure have the following advantages.

According to the resistance valve of the present disclosure, the multi-layer spiral plastic pipe accommodating space is formed by the fitting of the inner cylinder and the outer cylinder, the clamping length of the plastic pipe is increased by means of spiral winding, and appropriate resistance is provided to the plastic pipe through radial deformation of the cylinder, so as to provide back pressure required for the operation of the pump connected to the pipe, thereby reducing the impact on the flow medium in the plastic pipe.

The resistance valve of the present disclosure changes the pressure not only by changing a single parameter of the pipe radius, but by simultaneously changing two parameters of the pipe length and the flow cross-sectional area of the pipe, such that the adjustable pressure range and pressure adjustment precision can be improved.

The resistance valve described in the present disclosure provides pressure in the radial direction, which can provide a certain resistance without flattening the plastic pipe, such that blood damage caused by non-physiological shear stress resulting from compression can be avoided to a certain extent.

The present disclosure is provided with a relatively long plastic pipe accommodating space, which can store a relatively large amount of liquid when used as the resistance valve, so as to replace two members including the resistance valve and the liquid reservoir in the loop, such that the loop configuration can be simplified to a certain extent.

### BRIEF DESCRIPTION OF THE DRAWINGS

In order to facilitate a clear understanding of contents of the present disclosure, the present disclosure will be further illustrated in detail below according to specific embodiments of the present disclosure in conjunction with the drawings. In the drawings:

FIG. 1 is a schematic diagram of an overall structure of a resistance valve according to the present disclosure;

FIG. 2 is a schematic diagram of an internal sectional structure of a resistance valve according to the present disclosure;

FIG. 3 is a schematic diagram of an exploded structure of a resistance valve according to the present disclosure;

FIG. 4 is a schematic structural diagram of an inner cylinder and a bottom plate according to the present disclosure;

FIG. 5 is a schematic structural diagram of a sliding block according to the present disclosure;

FIG. 6 is a schematic structural diagram of a rotating shaft according to the present disclosure;

FIG. 7 is a schematic structural diagram of an outer cylinder according to the present disclosure;

FIG. 8 is a schematic structural diagram of a cover plate according to the present disclosure; and

FIG. 9 is a schematic structural diagram of an outer cylinder fixing ring according to the present disclosure.

Description of reference numerals: 1, bottom plate; 11, outer cylinder lower positioning block; 12, outer cylinder fixing ring slot; 13, retaining ring; 14, connection limiting rod; 2, inner cylinder; 21, deformation chute; 22, outer spiral groove; 23, deformation groove; 24, inner cylinder positioning groove; 3, outer cylinder; 31, inner spiral groove; 32, access opening; 33, snap-fit structure; 34, outer cylinder positioning groove; 4, outer cylinder fixing ring; 41, fixing block; 42, rotating handle; 43, Z-shaped slot; 5, sliding block; 51, inclined surface; 52, trapezoidal nut; 53, sliding block cover plate; 6, rotating shaft; 61, lower bushing; 62, bearing; 63, bearing cover plate; 64, upper bushing; 65, handle; 7, cover plate; 71, rotating shaft hole; 72, connecting rod hole; 73, inner cylinder positioning block; 74, outer cylinder upper positioning block; 8, plastic pipe accommodating space; and 9, wing screw.

### DETAILED DESCRIPTION OF THE PREFERRED EMBODIMENTS

The present disclosure will be further described below with reference to the drawings and specific embodiments, such that those skilled in the art can better understand and implement the present disclosure. However, the embodiments should not be construed as limiting the present disclosure.

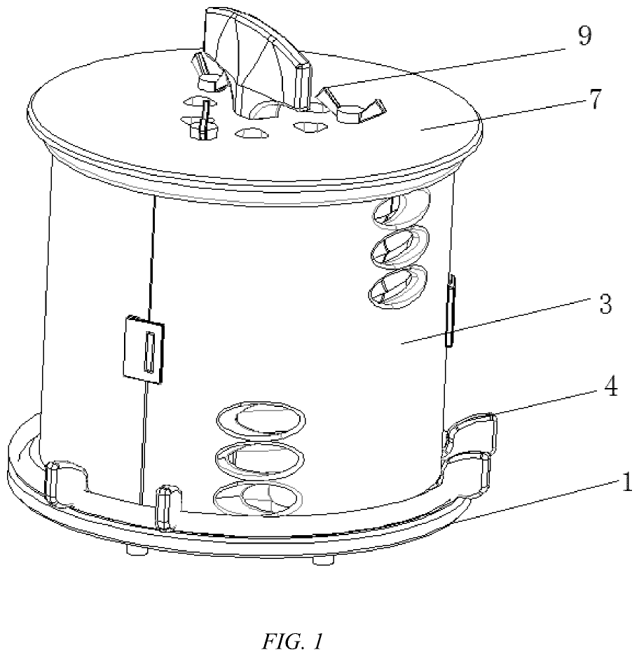

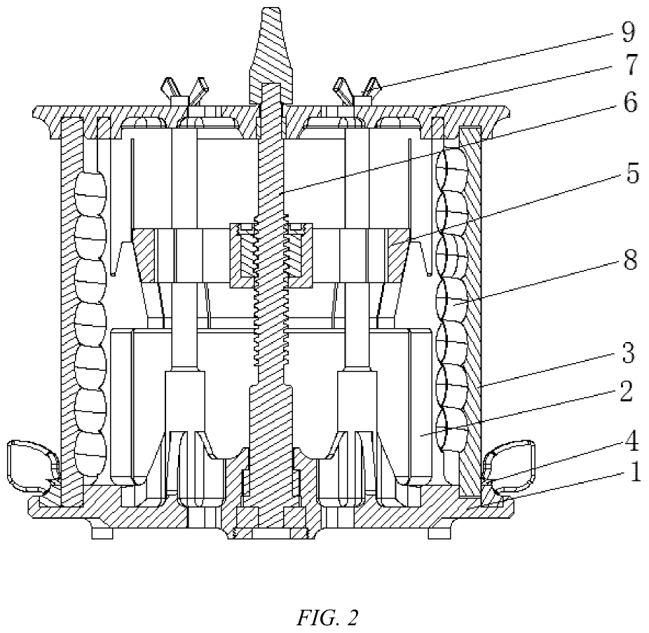

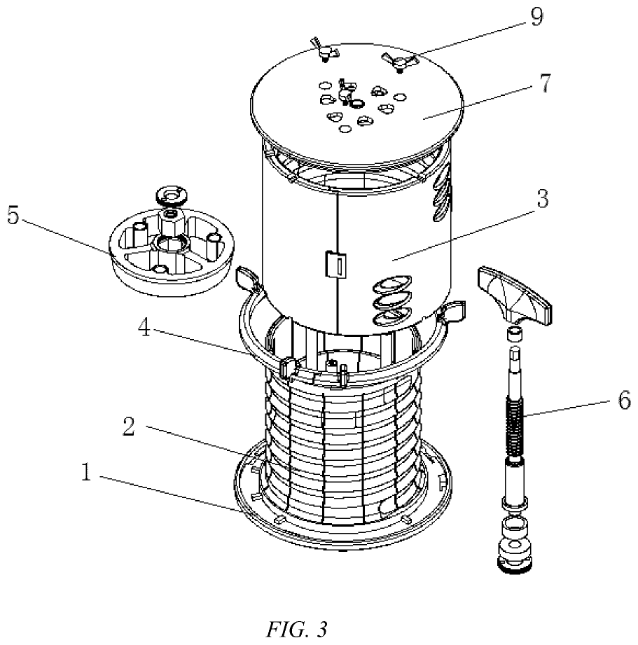

Referring to FIGS. 1-3, disclosed in the present disclosure is a resistance valve, which includes: a bottom plate 1, an inner cylinder 2 provided on the bottom plate 1, an outer cylinder 3 sleeved outside the inner cylinder 2, an outer cylinder fixing ring 4 enabling the outer cylinder 3 to be in locked connection to the bottom plate 1, a sliding block 5 provided in the inner cylinder 2, a rotating shaft 6 driving the sliding block 5 to slide in the inner cylinder 2, and a cover plate 7 snap-fitted onto the inner cylinder 2 and the outer cylinder 3. The inner cylinder 2 is a cylindrical structure, the inner cylinder 2 can deform outward along a radial direction, and an inclined deformation chute 21 is provided inside the inner cylinder 2. The sliding block 5 has an inclined surface 51 matching the deformation chute 21. The rotating shaft 6 is provided in the inner cylinder 2, the rotating shaft 6 extends through the sliding block 5 and is in thread connection to the sliding block 5, and the rotating shaft 6 rotates to drive the sliding block 5 to slide along an axial extension direction of the rotating shaft 6. The sliding block 5 slides in the inner cylinder 2 along an extension direction of the deformation chute 21, the inclined surface 51 of the sliding block 5 abuts against the deformation chute 21, and the sliding block 5 pushes the inner cylinder 2 to deform outward along the radial direction. A semicircular outer spiral groove 22 is formed on an outer wall of the inner cylinder 2, a semicircular inner spiral groove 31 is formed on an inner wall of the outer cylinder 3, and the inner spiral groove 31 is matched with the outer spiral groove to form a multi-layer spiral plastic pipe accommodating space 8.

The plastic pipe is wound between the inner cylinder 2 and the outer cylinder 3 along the multi-layer spiral plastic pipe accommodating space 8, thereby providing back pressure by frictional resistance loss along the plastic pipe and local resistance at bends. Meanwhile, the sliding block 5 pushes the inner cylinder 2 to deform radially, so as to clamp the plastic pipe in a tapered expansion-contraction manner to further supplement the back pressure, which avoids blood damage caused by a localized constriction structure. With the embodiment described above, the pressure is changed not only by changing a single parameter of the pipe radius, but by simultaneously changing two parameters of the pipe length and the flow cross-sectional area of the pipe, such that the adjustable pressure range and pressure adjustment precision can be improved. The pressure in the radial direction is provided by the radial deformation of the inner cylinder 2, which can provide a certain resistance without flattening the plastic pipe, such that blood damage caused by non-physiological shear stress resulting from compression can be avoided to a certain extent. Moreover, the resistance valve of this embodiment is provided with a relatively long plastic pipe accommodating space 8, which can store a relatively large amount of liquid when used as the resistance valve, so as to replace two members including the resistance valve and the liquid reservoir in the loop, such that the loop configuration can be simplified to a certain extent.

According to Poiseuille's law in fluid mechanics, as expressed by the formula: Q=π×r\hat{ }4×Δp/(8 ηL), the flow resistance (R) is defined as: 8 ηL/(πr\hat{ }4).

When the flow rate Q, the liquid viscosity coefficient η, and the pipe length L are constant, the flow resistance R is inversely proportional to the fourth power of the pipe radius r. This indicates that the pipe radius has a very large impact on the flow resistance, and therefore the conventional resistance valve relying only on changing the radius of plastic pipe exhibit limited adjustment precision.

When the flow rate Q, the fluid viscosity coefficient η, and the pipe radius r are constant, the pipe length L is directly proportional to the flow resistance R. This indicates that the back pressure can be provided by extending the pipe length.

The technical solution of this embodiment is derived from the above analysis of Poiseuille's law. By compressing the plastic pipe integrally in its length direction by radial deformation of the inner cylinder 2, the formation of a constriction structure with an excessively small cross-sectional area by localized compression is avoided. In addition, by means of spiral winding, the pipe length can be further increased, which can also provide back pressure required for the pipe in the loop, thereby reducing the impact on the flowing medium in the pipe.

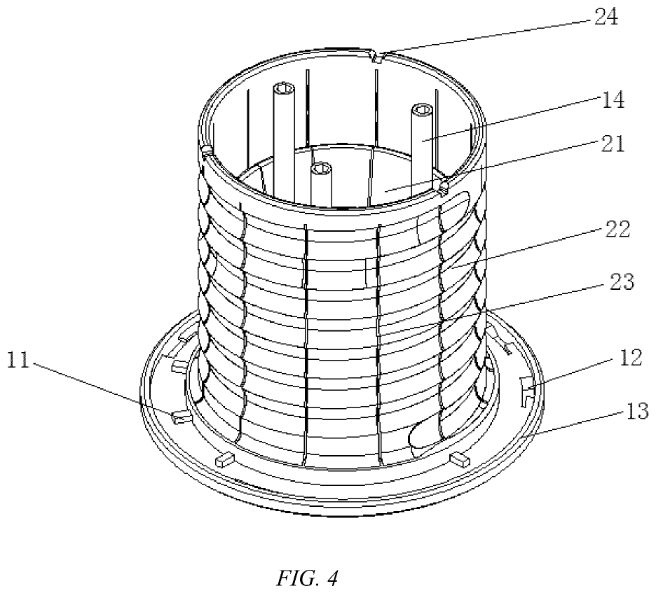

Referring to FIG. 4, in this embodiment, the inner cylinder 2 and the bottom plate 1 are integrally formed. In order to achieve the deformation of the inner cylinder 2 along the radial direction, a plurality of deformation grooves 23 are formed on the side wall of the inner cylinder 2, the deformation grooves 23 are arranged along the axial extension direction of the inner cylinder 2, and the plurality of deformation grooves 23 are evenly distributed around the circumference of the inner cylinder 2. When the side wall of the inner cylinder 2 is compressed by the sliding block 5, the deformation grooves 23 expand, thereby increasing the diameter of the inner cylinder 2.

Specifically, in this embodiment, the outer cylinder 3 is sleeved outside the inner cylinder 2, and the outer cylinder 3 also needs to be connected to the bottom plate 1. Therefore, the bottom plate 1 is provided with an outer cylinder lower positioning block 11 for limiting the outer cylinder 3. The outer cylinder 3 is placed on the bottom plate 1 and is in locked connection to the bottom plate 1 by the outer cylinder fixing ring 4. Therefore, the bottom plate 1 is further provided with an outer cylinder fixing ring slot 12 and a retaining ring for limiting the outer cylinder fixing ring 4.

Specifically, in this embodiment, during assembly, since the inner spiral groove 31 on the inner wall of the outer cylinder 3 needs to be matched with the outer spiral groove on the outer wall of the inner cylinder 2, only one positional relationship exists between the outer cylinder 3 and the inner cylinder 2. In order to ensure the accuracy of the sleeving position of the outer cylinder 3, the bottom plate 1 is provided with a plurality of outer cylinder lower positioning blocks 11, and the plurality of outer cylinder lower positioning blocks 11 are unevenly distributed, such that only one positional connection relationship exists between the outer cylinder 3 and the bottom plate 1.

Specifically, in this embodiment, the cover plate 7 is snap-fitted onto the inner cylinder 2. Therefore, an inner cylinder positioning groove 24 is provided at a connection position between the upper edge of the inner cylinder 2 and the cover plate 7. When the cover plate 7 is snap-fitted onto the inner cylinder 2, the position of the cover plate 7 is limited by the inner cylinder positioning groove 24.

Specifically, in order to achieve the connection between the cover plate 7 and the inner cylinder 2, in this embodiment, the bottom plate 1 is further provided with a connection limiting rod 14. The connection limiting rod 14 is provided in the inner cylinder 2, the connection limiting rod 14 extends along a direction of the cover plate 7, and the cover plate 7 is in locked connection to the connection limiting rod 14 by a wing screw 9, thereby achieving the fixation of the cover plate 7. In this embodiment, the connection limiting rod 14 functions to fix the cover plate 7. Moreover, since the connection limiting rod 14 is provided in the inner cylinder 2, and the connection limiting rod 14 extends through the sliding block 5, enabling the sliding block 5 to slide in the inner cylinder 2 along an extension direction of the connection limiting rod 14, the connection limiting rod 14 also functions to limit and guide the sliding block 5.

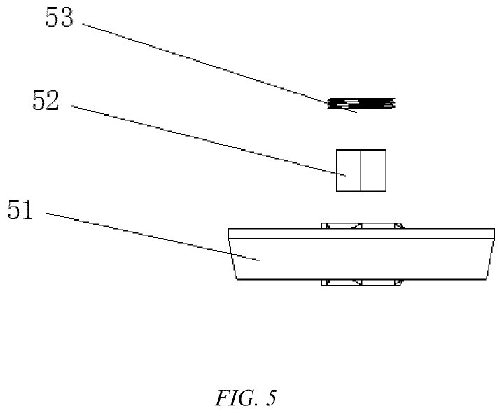

Referring to FIG. 5, in this embodiment, in order to achieve the threaded connection between the sliding block 5 and the rotating shaft 6, a trapezoidal nut 52 is embedded in the central position of the sliding block 5. The trapezoidal nut 52 is in limited fixation in the sliding block 5 by a sliding block cover plate 53, and the sliding block cover plate 53 is in threaded connection to the sliding block 5.

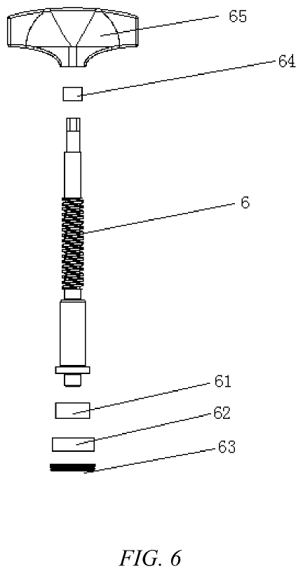

Referring to FIG. 6, in this embodiment, in order to achieve the connection between the rotating shaft 6 and the bottom plate 1, a lower bushing 61 and a bearing 62 are provided at a connection position between the rotating shaft 6 and the bottom plate 1. One end of the lower bushing 61 is sleeved outside the rotating shaft 6 and is connected to the rotating shaft 6, and the other end of the bushing is inserted into the bearing 62. The bearing 62 is embedded in the bottom plate 1, and the bearing 62 is fixed in the bottom plate 1 by a bearing cover plate 63. The bearing cover plate 63 is in threaded connection to the bottom plate 1. As such, the connection between the rotating shaft 6 and the bottom plate 1 is achieved by the bushing and the bearing 62, and meanwhile, the rotating shaft 6 can be ensured to rotate relative to the bottom plate 1. In order to achieve the connection between the rotating shaft 6 and the cover plate 7, an upper bushing 64 is provided at a connection position between the rotating shaft 6 and the cover plate 7.

Specifically, in order to facilitate the driving of the rotation of the rotating shaft 6, a handle 65 is further provided at an end of the rotating shaft 6.

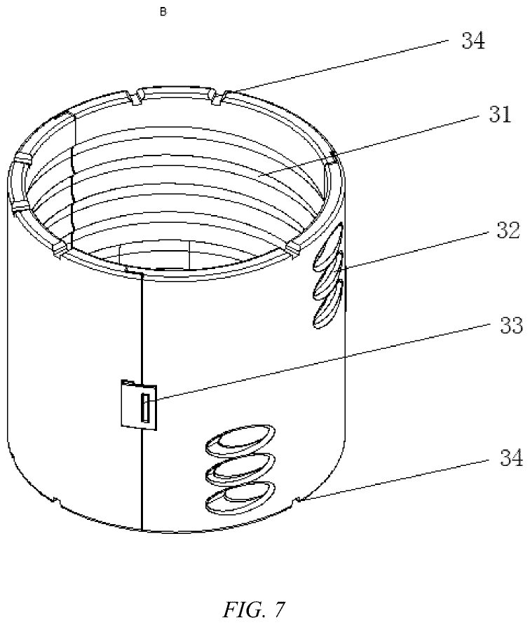

Referring to FIG. 7, a plurality of access openings 32 are formed on the side wall of the outer cylinder 3, the plurality of access openings 32 are correspondingly in communication with different layers of plastic pipe accommodating spaces 8, respectively, and the plastic pipe enters and exits from the access openings 32. In this embodiment, a total of seven layers of spiral plastic pipe accommodating spaces 8 are provided, and correspondingly, six access openings 32 are formed on the side wall of the outer cylinder 3. The six access openings 32 are located in layers 1, 2, 3, 5, 6, and 7 of plastic pipe accommodating spaces 8, respectively, such that the plastic pipe can be wound in the plastic pipe accommodating spaces 8 according to a specified number of turns. For example, when the required number of winding turns for the plastic pipe is 3, the two ends of the plastic pipe can pass through the access openings 32 in layers 3 and 5, respectively; when the required number of winding turns for the plastic pipe is 5, the two ends of the plastic pipe can pass through the access openings 32 in layers 2 and 6, respectively; when the required number of winding turns for the plastic pipe is 7, the two ends of the plastic pipe can pass through the access openings 32 in layers 1 and 7, respectively.

In other embodiments, different numbers of layers of plastic pipe accommodating spaces 8 and access openings 32 corresponding to different layer positions can be provided according to actual use requirements, so as to achieve a combination of more winding requirements.

Specifically, in order to facilitate detachment and mounting, in this embodiment, the outer cylinder 3 is configured as a cylindrical structure formed by splicing a plurality of arc-shaped sheets end to end, with a snap-fit structure 33 provided between two adjacent arc-shaped sheets.

Specifically, the outer cylinder 3 is placed on the bottom plate 1 first, and then the cover plate 7 is snap-fitted onto the outer cylinder 3. In order to achieve the connection between the outer cylinder 3 and the bottom plate 1 and the cover plate 7, outer cylinder positioning grooves 34 are provided at connection positions between the outer cylinder 3 and the bottom plate 1 and the cover plate 7.

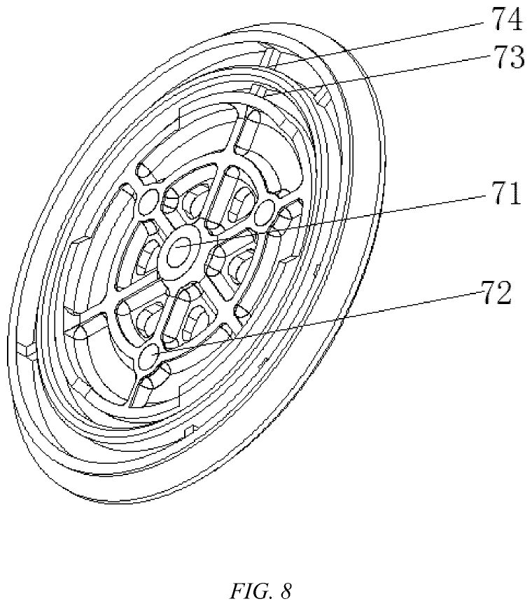

Referring to FIG. 8, in this embodiment, the rotating shaft 6 protrudes beyond the inner cylinder 2, and the protruding portion of the rotating shaft 6 passes through the cover plate 7. Therefore, a rotating shaft hole 71 is formed on the cover plate 7 for the rotating shaft 6 to pass through. In addition, in this embodiment, the connection limiting rod 14 also needs to pass through the cover plate 7, and then the cover plate 7 is in locked connection to the connection limiting rod 14 by a bolt. Therefore, a connecting rod hole 72 is further formed on the cover plate 7.

Specifically, in order to achieve the connection between the cover plate 7 and the inner cylinder 2 and the outer cylinder 3, the cover plate 7 is further provided with an inner cylinder positioning block 73 and an outer cylinder upper positioning block 74. The inner cylinder positioning block 73 is matched with the inner cylinder positioning groove 24 on the inner cylinder 2 in position, and the outer cylinder upper positioning block 74 is matched with the outer cylinder positioning groove 34 on the outer cylinder 3 in position.

Specifically, in this embodiment, during assembly, in order to ensure that only one positional relationship exists between the cover plate 7 and the outer cylinder 3 and the inner cylinder 2, the cover plate 7 is provided with a plurality of outer cylinder upper positioning blocks 84. The plurality of outer cylinder upper positioning blocks 84 are unevenly distributed, such that only one positional connection relationship exists between the cover plate 7 and the outer cylinder 3.

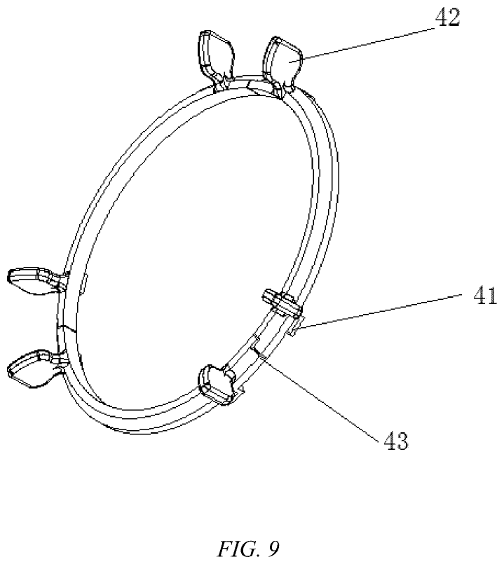

Referring to FIG. 9, in this embodiment, the outer cylinder 3 is in locked fixation to the bottom plate 1 by the outer cylinder fixing ring 4. Therefore, the outer cylinder fixing ring 4 is provided with a fixing block 41, and the fixing block 41 is matched with an outer cylinder fixing ring slot 12 formed on the bottom plate 1. The fixing block 41 can be inserted into the outer cylinder fixing ring slot 12 by rotating the outer cylinder fixing ring 4, thereby achieving a quick connection between the outer cylinder fixing ring 4 and the bottom plate 1.

Specifically, in order to facilitate the rotation of the outer cylinder fixing ring 4, the outer cylinder fixing ring 4 is further provided with a rotating handle 42.

Specifically, in this embodiment, in order to facilitate the mounting of the outer cylinder fixing ring 4, the outer cylinder fixing ring 4 is also formed by splicing a plurality of arc-shaped ring bodies end to end, with two adjacent arc-shaped ring bodies connected by a Z-shaped slot 43.

The mounting process of the resistance valve in this embodiment is as follows:
1. Preparation of the sliding block 5: the trapezoidal nut 52 is placed into the central groove of the sliding block 5, and the sliding block cover plate 53 is screwed into the threaded portion of the central groove, so as to fix the trapezoidal nut 52 in the sliding block 5.
2. Mounting the rotating shaft 6: the lower bushing 61 is placed into the slot of the shaft end on the lower side of the base, the bearing 62 is connected to the rotating shaft 6, then the assembled rotating shaft 6 is inserted through the opening on the lower side of the base, the bearing 62 is fixedly connected to the base, and finally, the bearing cover plate 63 is screwed into the threaded portion of the central groove.
3. The sliding block 5 is screwed into the rotating shaft 6.
4. The upper bushing 64 is placed into the rotating shaft hole 71 of the cover plate 7.
5. The plastic pipe is wound into the outer spiral groove of the inner cylinder 2 according to the specified number of turns (3/5/7 turns).
6. The outer cylinder 3 is placed on the base according to the arrangement of the outer cylinder lower positioning blocks 11, with the two ends of the plastic pipe passing through the access openings.
7. The outer cylinder fixing ring 4 is mounted into the corresponding position of the base, and then the outer cylinder fixing ring 4 is rotated to a locking position by using the rotating handle 42 to achieve the locking of the outer cylinder 3.
8. The cover plate 7 is snap-fitted onto the inner cylinder 2 and the outer cylinder 3 according to the arrangement of the inner cylinder positioning grooves 24 and the outer cylinder positioning grooves 34.
9. The cover plate 7 is in locked connection to the connection limiting rod 14 by using wing screws 9.
10. The handle 65 is mounted onto the rotating shaft 6.

The using method of the resistance valve of this embodiment is as follows:

After the mounting, the handle 65 is turned clockwise to drive the sliding block 5 to move downward, such that the inner cylinder 2 deforms outward along the radial direction to achieve the effect of clamping the plastic pipe, thereby achieving the purpose of providing considerable resistance to the circuit.

After the clamping, the handle 65 is turned counterclockwise to drive the sliding block 5 to move upward, such that the inner cylinder 2 deforms inward along the radial direction to achieve the effect of loosening the plastic pipe, thereby achieving the purpose of reducing the resistance of the circuit.

Specifically, in this embodiment, the bottom plate 1, the inner cylinder 2, the outer cylinder 3, the outer cylinder fixing ring 4, the sliding block 5, and the cover plate 7 can all be processed by adopting technologies such as 3D printing and injection molding.

In the prepared sample, the inner cylinder 2, which is required to withstand both bending deformation and substantial radial forces, is prepared by 3D printing using high-toughness nylon. For the sliding block 5, which is embedded with a trapezoidal nut 52 and needs to withstand radial pressure, nylon reinforced with glass fiber can be selected for its preparation by 3D printing.

Considering that the torsional strength of the rotating shaft 6 prepared by 3D printing is insufficient, the rotating shaft 6 needs to be made of steel to meet the torsional strength requirement.

It is obvious that the above embodiments are merely examples for clear illustration and are not intended to limit implementations. Various changes and modifications can be made by those of ordinary skill in the art on the basis of the above description. It is unnecessary and impossible to exhaust all the embodiments herein. Obvious changes or modifications derived therefrom still fall within the protection scope of the present disclosure.

1. A resistance valve configured to provide resistance to a liquid in a plastic pipe, comprising:
an inner cylinder, wherein the inner cylinder is configured as a cylindrical structure, the inner cylinder can deform outward along a radial direction, a semi-elliptical outer spiral groove is formed on an outer wall of the inner cylinder, and an inclined deformation chute is provided inside the inner cylinder;
a sliding block provided in the inner cylinder, wherein the sliding block has an inclined surface matching the deformation chute, the sliding block slides in the inner cylinder along an extension direction of the deformation chute, the inclined surface of the sliding block abuts against the deformation chute, and the sliding block pushes the inner cylinder to deform outward along the radial direction;
a rotating shaft provided in the inner cylinder, wherein the rotating shaft extends through the sliding block and is in threaded connection to the sliding block, and the rotating shaft rotates to drive the sliding block to slide along an axial extension direction of the rotating shaft; and
an outer cylinder sleeved outside the inner cylinder, wherein a semi-elliptical inner spiral groove is opened on an inner wall of the outer cylinder, and the inner spiral groove is matched with the outer spiral groove to form a multi-layer spiral plastic pipe accommodating space.

2. The resistance valve according to claim 1, wherein a plurality of deformation grooves are opened on a side wall of the inner cylinder, the deformation grooves are arranged along an axial extension direction of the inner cylinder, and the plurality of deformation grooves are evenly distributed around a circumference of the inner cylinder.

3. The resistance valve according to claim 1, wherein a plurality of access openings are formed on a side wall of the outer cylinder, the plurality of access openings are correspondingly in communication with different layers of plastic pipe accommodating spaces, respectively, and the plastic pipe enters and exits from the access openings.

4. The resistance valve according to claim 1, wherein the outer cylinder is a cylindrical structure formed by splicing a plurality of arc-shaped sheets end to end, with a snap-fit structure being provided between two adjacent arc-shaped sheets.

5. The resistance valve according to claim 1, wherein the rotating shaft partially protrudes from the inner cylinder, and a handle is provided at an end of the rotating shaft protruding from the inner cylinder.

6. The resistance valve according to claim 1, further comprising:
a bottom plate, wherein the inner cylinder and the outer cylinder are both provided on the bottom plate, the inner cylinder and the bottom plate are integrally formed, and the outer cylinder is snap-fitted onto the bottom plate;
an outer cylinder fixing ring sleeved outside the outer cylinder, wherein a retaining ring for limiting the outer cylinder fixing ring is formed on the bottom plate, and the outer cylinder fixing ring enables the outer cylinder to be in locked connection to the bottom plate;
a cover plate provided corresponding to the bottom plate and snap-fitted onto the inner cylinder and the outer cylinder, wherein a rotating shaft hole is opened on the cover plate; and
a connection limiting rod provided on the bottom plate and extending toward the cover plate, wherein the cover plate is in locked connection to the connection limiting rod by a bolt.

7. The resistance valve according to claim 6, wherein
the outer cylinder fixing ring is provided with a fixing block, and an outer cylinder fixing ring slot is opened on the bottom plate; the outer cylinder fixing ring slot is provided corresponding to the fixing block in position, and the fixing block can be inserted into the outer cylinder fixing ring slot by rotating the outer cylinder fixing ring;
the outer cylinder fixing ring is further provided with a rotating handle.

8. The resistance valve according to claim 6, wherein the cover plate is further provided with an inner cylinder positioning block, and an inner cylinder positioning groove matching the inner cylinder positioning block is provided at a connection position between the inner cylinder body and the cover plate.

9. The resistance valve according to claim 6, wherein the bottom plate is provided with an outer cylinder lower positioning block and the cover plate is provided with an outer cylinder upper positioning block, and outer cylinder positioning grooves matching the outer cylinder lower positioning block and the outer cylinder upper positioning block are respectively provided at a connection position between the outer cylinder and the bottom plate, and a connection position between the outer cylinder and the cover plate.

10. The resistance valve according to claim 9, wherein the bottom plate and the cover plate are respectively provided with a plurality of outer cylinder lower positioning blocks and outer cylinder upper positioning blocks, and the plurality of outer cylinder lower positioning blocks and outer cylinder upper positioning blocks are unevenly distributed.
